SEQUENCE LISTING (5 sequences)

SEQ ID NO: 1 - DNA, 25 bp, Artificial Sequence
  Description of Artificial Sequence Synthetic primer
gatctctggtctttcccagcgtcaa

SEQ ID NO: 2 - DNA, 27 bp, Artificial Sequence
  Description of Artificial Sequence Synthetic primer
gagccccgtccaaggacgtaaaatgaa

SEQ ID NO: 3 - DNA, 27 bp, Artificial Sequence
  Description of Artificial Sequence Synthetic primer
gagccccgtccaaggacgttaaaagaa

SEQ ID NO: 4 - DNA, 24 bp, Artificial Sequence
  Description of Artificial Sequence Synthetic primer
tattgaagtcaggccacttgtgcc

SEQ ID NO: 5 - DNA, 30 bp, Artificial Sequence
  Description of Artificial Sequence Synthetic probe
aaggactagaggttagaggagaccccccgc

(19) United States
(12) Patent Application Publication
(10) Pub. No.: US 2016/0168186 A1
(43) Pub. Date: Jun. 16, 2016

(54) Method of Treating Dengue Fever

(71) Applicant: RIBOSCIENCE LLC, Palo Alto, CA (US)

(72) Inventors: Hassan Javanbakht, New York, NY (US); Klaus G. Klumpp, West Chester, PA (US); Suping Ren, Livingston, NJ (US); Zhuming Zhang, Hillsborough, NJ (US)

(21) Appl. No.: 14/879,463

(22) Filed: Oct. 9, 2015

Related U.S. Application Data
(63) Continuation of application No. 13/488,802, filed on Jun. 5, 2012, now abandoned.
(60) Provisional application No. 61/495,472, filed on Jun. 10, 2011.

(51) Int. Cl. C07H 19/20; C07H 19/10; A61K 31/7072; A61K 45/06; A61K 31/7076

(52) U.S. Cl. CPC C07H 19/20; A61K 45/06; A61K 31/7076; A61K 31/7072; C07H 19/10

(57) ABSTRACT

The application provides methods for treatment or prophylaxis of dengue virus mediated diseases with compounds of Formula I

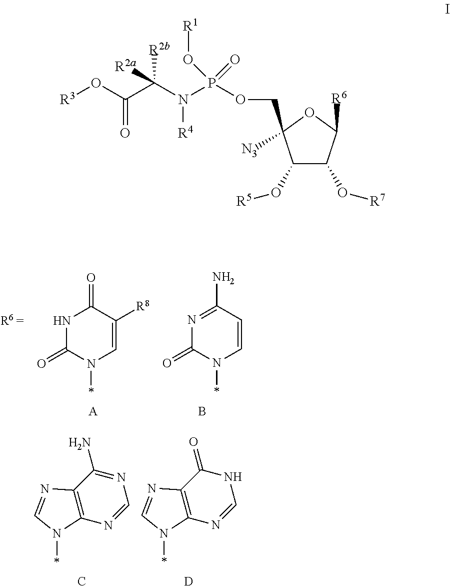

wherein R¹, R^{2a}, R^{2b}, R³, R⁴, R⁵ R⁶, R^{8a}, R⁹ and R¹⁰ are as defined herein. The application further discloses methods for treatment or prophylaxis of dengue virus mediated diseases with pharmaceutical compositions comprising compounds of Formula I.

### CROSS-REFERENCE TO RELATED APPLICATIONS

This application claims priority to co-pending application Ser. No. 13/488,808, filed Jun. 6, 2012, and entitled “Method of Treating Dengue Fever,” which itself claims priority to application Ser. No. 61/495,472 filed on Jun. 10, 2011, and entitled Method of Treating Dengue Fever.” The contents of the co-pending applications are fully incorporated herein by reference.

### FIELD OF THE INVENTION

The present application provides nucleoside compounds of Formulae I for the treatment of dengue fever (DF). The present application provides methods of treatment of dengue fever using the nucleoside compounds of Formula I.

Dengue fever is an acute febrile disease caused by one of four closely related virus serotypes (DEN-I, DEN-2, DEN-3, and DEN-4). Dengue fever is classified based on its clinical characteristics into classical dengue fever, or the more severe forms, dengue hemorrhagic fever syndrome (DHF), and dengue shock syndrome (DSS). Recovery from infection from one serotype produces life-long immunity to that particular serotype, but provides only short-lived and limited protection against any of the other serotypes. Dengue is a member of the Flaviviridae family which are enveloped, positive-sense RNA viruses whose human pathogens also include West Nile virus (WNV), yellow fever virus (YFV), Japanese encephalitis virus (JEV), and tick-borne encephalitis virus (TBEV) among others.

Dengue transmission is via the bite of an infected Aedes aegypti mosquito which is found in tropical and sub-tropical regions around the world.

Each year regional epidemics of dengue cause significant morbidity and mortality, social disruption and substantial economic burden on the societies affected both in terms of hospitalization and mosquito control. Dengue is considered by the World Health Organization (WHO) to be the most important arthropod-borne viral disease with an estimated 50 million cases of dengue infection, including 500,000 DHF cases and 24,000 deaths worldwide each year. WHO estimates that forty percent of the world's population (2.5 billion people) are at risk for DF, DHF, and DSS. Dengue is also a NIAID Category A pathogen and in terms of bio-defense, represents a significant threat to United States troops overseas. Dengue is an emerging threat to North America with a dramatic increase in severe disease in the past 25 years including major epidemics in Cuba and Venezuela, and outbreaks in Texas and Hawaii.

Failure to control the mosquito vector and increases in long-distance travel have contributed to the increase and spread of dengue disease. The characteristics of dengue as a viral hemorrhagic fever virus (arthropod-borne, widely spread, and capable of inducing a great amount of cellular damage and eliciting an immune response that can result in severe hemorrhage, shock, and death) makes this virus a unique threat to deployed military personnel around the world as well as to travelers to tropical regions. Preparedness for both biodefense and for the public health challenges posed by dengue will require the development of new vaccines and antiviral therapeutics.

Dengue causes several illnesses with increasing severity being determined in part by prior infection with a different serotype of the virus. Classic dengue fever (DF) begins 3-8 days after the bite of an infected mosquito and is characterized by sudden onset of fever, headache, back pain, joint pain, a measles-like rash, and nausea and vomiting. DF is frequently referred to as “breakbone” fever due to these symptoms. The disease usually resolves after two weeks but a prolonged recovery with weakness and depression is common.

The more severe form of the disease, dengue hemorrhagic fever (DHF) has a similar onset and early phase of illness as dengue fever. However, shortly after onset the disease is characterized by high fever, enlargement of the liver, and hemorrhagic phenomena such as bleeding from the nose, mouth, and internal organs due to vascular permeability. In dengue shock syndrome (DSS) circulatory failure and hypovolaemic shock resulting from plasma leakage occur and can lead to death in 12-24 hours without plasma replacement. The case fatality rate of DHF/DSS can be as high as 20% without treatment. DHF has become a leading cause of hospitalization and death among children in many countries with an estimated 500,000 cases requiring hospitalization each year and a case fatality rate of about 5%.

The pathogenesis of DHF/DSS is still being studied but is thought to be due in part to an enhancement of virus replication in macrophages by heterotypic antibodies, termed antibody-dependent enhancement (ADE). During a secondary infection, with a different serotype of dengue virus, cross-reactive antibodies that are not neutralizing form virus-antibody complexes that are taken into monocytes and Langerhans cells (dendritic cells) and increase the number of infected cells. This leads to the activation of cytotoxic lymphocytes which can result in plasma leakage and the hemorrhagic features characteristic of DHF and DSS. This antibody-dependent enhancement of infection is one reason why the development of a successful vaccine has proven to be so difficult. Although less frequent, DHF/DSS can occur after primary infection, so virus virulence and immune activation are also believed to contribute to the pathogenesis of the disease.

Dengue is endemic in more than 100 countries in Africa, the Americas, the Eastern Mediterranean, South-east Asia and the Western Pacific. During epidemics, attack rates can be as high as 80-90% of the susceptible population. All four serotypes of the virus are emerging worldwide, increasing the number of cases of the disease as well as the number of explosive outbreaks. In 2002 for example, there were 1,015,420 reported cases of dengue in the Americas alone with 14,374 cases of DHF, which is more than three times the number of dengue cases reported in the Americas in 1995.

The dengue genome, approximately 11 kb in length, consists of a linear, single stranded, infectious, positive sense RNA that is translated as a single long polyprotein.

The genome is composed of seven nonstructural (NS) protein genes and three structural protein genes which encode the nucleocapsid protein (C), a membrane-associated protein (M), and an envelope protein (E). The nonstructural proteins are involved in viral RNA replication viral assembly, and the inflammatory components of the disease. The structural proteins are involved mainly in viral particle formation. The precursor polyprotein is cleaved by cellular proteinases to separate the structural proteins, while a virus-encoded proteinase cleaves the nonstructural region of the polyprotein. The genome is capped and does not have a poly (A) tail at the 3′ end but instead has a stable stem-loop structure necessary for stability and replication of the genomic RNA. The virus binds to cellular receptors via the E protein and undergoes receptor-mediated endocytosis followed by low-pH fusion in lysosomes.

The viral genome is then uncoated and translated into the viral precursor polyprotein. Co- and posttranslational proteolytic processing separates the structural and nonstructural proteins. The RNA-dependent RNA polymerase along with cofactors synthesizes the minus-strand RNA which serves as a template for the synthesis of the progeny plus-strand RNA.

Viral replication is membrane associated. Following replication, the genome is encapsidated, and the immature virus, surrounded by a lipid envelope buds into the lumen. The envelope proteins become glycosylated and mature viruses are released outside the cell. Essential stages or process during the virus life cycle would be possible targets for inhibition from an antiviral drug and include binding of the virus to the cell through the E protein, uptake of the virus into the cell, the capping mechanism, the viral proteinase, the viral RNA-dependent RNA polymerase, and the viral helicase.

Current management of dengue virus-related disease relies solely on vector control. There are no approved antivirals or vaccines for the treatment or prevention of dengue. Ribavirin, a guanosine analogue, has been shown to be effective against a range of RNA virus infections and works against dengue in tissue culture by inhibiting the dengue 2′-O-methyltransferase NS5 domain. However, ribavirin did not show protection against dengue in a mouse model or a rhesus monkey model, instead it induced anemia and thrombocytosis.

While there are no currently available approved vaccines, multivalent dengue vaccines have shown some limited potential in humans. However, vaccine development is difficult due to the presence of four distinct serotypes of the virus which each cause disease. Vaccine development also faces the challenge of ADE where unequal protection against the different strains of the virus could actually increase the risk of more serious disease. Therefore there is a need for antiviral drugs that target all of the serotypes of dengue. An antiviral drug administered early during dengue infection that inhibits viral replication would prevent the high viral load associated with DHF and be an attractive strategy in the treatment and prevention of disease. An antiviral drug that inhibits viral replication could be administered prior to travel to a dengue endemic region to prevent acquisition of disease, or for those that have previously been exposed to dengue, could prevent infection by another serotype of virus and decrease the chance of life-threatening DHF and DSS. Having an antiviral drug would also aid vaccine development by having a tool at hand to treat complications that may arise due to unequal immune protection against the different serotypes. Although a successful vaccine could be a critical component of an effective biodefense, the typical delay to onset of immunity, potential side-effects, cost, and logistics associated with large-scale civilian vaccinations against a low-threat risk agent suggest that a comprehensive biodefense include a separate rapid-response element.

There is a clear and long-felt need to develop effective therapeutics for treatment of dengue virus. Specifically, there is a need to develop compounds that are useful for treating dengue-infected patients and compounds that selectively inhibit dengue viral replication.

### SUMMARY OF THE INVENTION

The application provides a method for treating dengue fever comprising administering to a patient in need thereof a compound of Formula I

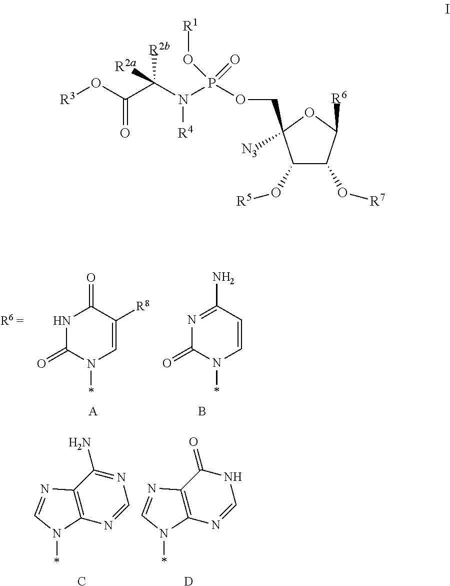

wherein:
R¹ is hydrogen, C₁₋₆haloalkyl, or aryl wherein said aryl is phenyl or naphthyl optionally substituted with one to three substituents independently selected from the group consisting of C₁₋₆alkyl, C₂₋₆alkenyl, C₂₋₆alkynyl, C₁₋₆alkoxy, halogen, C₁₋₆haloalkyl, —N(R^{1a})₂, C₁₋₆acylamino, —NHSO₂C₁₋₆alkyl, —SO₂N(R^{1a})₂, —SO₂C₁₋₆alkyl, —COR^{1b}, nitro and cyano;
R^{1a} is independently hydrogen or C₁₋₆alkyl;
R^{1b} is —OR^{1a} or —N(R^{1a})₂;
R^{2a} and R^{2b} are (i) independently selected from the group consisting of hydrogen, C₁₋₁₀alkyl, —(CH2)ᵣN(R^{1a})₂, C₁₋₆hydroxyalkyl, —CH₂SH, —(CH₂)S(O)ₚMe, —(CH2)₃NHC(═NH)NH₂, (1H-indol-3-yl)methyl, (1H-indol-4-yl)methyl, —(CH2)ₘC(═O)R^{1b}, aryl and aryl C₁₋₃alkyl, said aryl groups optionally substituted with a group selected from the group consisting of hydroxyl, C₁₋₁₀alkyl, C₁₋₆alkoxy, halogen, nitro and cyano; (ii) R^{2a} is hydrogen and R^{2b} and R⁴ together are (CH₂)₃; (iii) R^{2a} and R^{2b} together are (CH₂)ₙ; or, (iv) R^{2a} and R^{2b} both are C₁₋₆ alkyl;
R³ is hydrogen, C₁₋₁₀ alkyl, C₁₋₁₀ haloalkyl, aryl or aryl-C₁₋₃ alkyl wherein said aryl is phenyl;
R⁴ is hydrogen, C₁₋₃ alkyl, or R^{2b} and R⁴ together are (CH₂)₃;
R⁶ is A, B, C or D wherein R⁸ is hydrogen or C₁₋₃ alkyl;
R⁵ and R⁷ is independently selected from hydrogen, C(═O)C₁₋₆alkyl, C(═O)R^{1b};
m is 0 to 3;
n is 4 or 5;
p is 0 to 2; and
r is 1 to 6;
or pharmaceutically acceptable salts thereof.

### DETAILED DESCRIPTION OF THE INVENTION

### Definitions

The phrase “a” or “an” entity as used herein refers to one or more of that entity; for example, a compound refers to one or more compounds or at least one compound. As such, the terms “a” (or “an”), “one or more”, and “at least one” can be used interchangeably herein.

The phrase “as defined herein above” refers to the broadest definition for each group as provided in the Summary of the Invention or the broadest claim. In all other embodiments provided below, substituents which can be present in each embodiment and which are not explicitly defined retain the broadest definition provided in the Summary of the Invention.

As used in this specification, whether in a transitional phrase or in the body of the claim, the terms “comprise(s)” and “comprising” are to be interpreted as having an open-ended meaning That is, the terms are to be interpreted synonymously with the phrases “having at least” or “including at least”. When used in the context of a process, the term “comprising” means that the process includes at least the recited steps, but may include additional steps. When used in the context of a compound or composition, the term “comprising” means that the compound or composition includes at least the recited features or components, but may also include additional features or components.

As used herein, unless specifically indicated otherwise, the word “or” is used in the “inclusive” sense of “and/or” and not the “exclusive” sense of “either/or”.

The term “independently” is used herein to indicate that a variable is applied in any one instance without regard to the presence or absence of a variable having that same or a different definition within the same compound. Thus, in a compound in which R″ appears twice and is defined as “independently carbon or nitrogen”, both R″s can be carbon, both R″s can be nitrogen, or one R″ can be carbon and the other nitrogen.

When any variable occurs more than one time in any moiety or formula depicting and describing compounds employed or claimed in the present invention, its definition on each occurrence is independent of its definition at every other occurrence. Also, combinations of substituents and/or variables are permissible only if such compounds result in stable compounds.

The symbols “*” at the end of a bond or “ [symbol] ” drawn through a bond each refer to the point of attachment of a functional group or other chemical moiety to the rest of the molecule of which it is a part. Thus, for example:

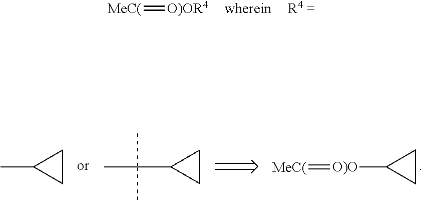

A bond drawn into ring system (as opposed to connected at a distinct vertex) indicates that the bond may be attached to any of the suitable ring atoms.

The term “optional” or “optionally” as used herein means that a subsequently described event or circumstance may, but need not, occur, and that the description includes instances where the event or circumstance occurs and instances in which it does not. For example, “optionally substituted” means that the optionally substituted moiety may incorporate a hydrogen atom or a substituent.

The phrase “optional bond” means that the bond may or may not be present, and that the description includes single, double, or triple bonds. If a substituent is designated to be a “bond” or “absent”, the atoms linked to the substituents are then directly connected.

The term “about” is used herein to mean approximately, in the region of, roughly, or around. When the term “about” is used in conjunction with a numerical range, it modifies that range by extending the boundaries above and below the numerical values set forth. In general, the term “about” is used herein to modify a numerical value above and below the stated value by a variance of 20%.

Certain compounds may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

Technical and scientific terms used herein have the meaning commonly understood by one of skill in the art to which the present invention pertains, unless otherwise defined. Reference is made herein to various methodologies and materials known to those of skill in the art. Standard reference works setting forth the general principles of pharmacology include Goodman and Gilman's The Pharmacological Basis of Therapeutics, 10^{th} Ed., McGraw Hill Companies Inc., New York (2001). Any suitable materials and/or methods known to those of skill can be utilized in carrying out the present invention. However, preferred materials and methods are described. Materials, reagents and the like to which reference are made in the following description and examples are obtainable from commercial sources, unless otherwise noted.

The definitions described herein may be appended to form chemically-relevant combinations, such as “heteroalkylaryl,” “haloalkylheteroaryl,” “arylalkylheterocyclyl,” “alkylcarbonyl,” “alkoxyalkyl,” and the like. When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” refers to an alkyl group having one to two phenyl substituents, and thus includes benzyl, phenylethyl, and biphenyl. An “alkylaminoalkyl” is an alkyl group having one to two alkylamino substituents. “Hydroxyalkyl” includes 2-hydroxyethyl, 2-hydroxypropyl, 1-(hydroxymethyl)-2-methylpropyl, 2-hydroxybutyl, 2,3-dihydroxybutyl, 2-(hydroxymethyl), 3-hydroxypropyl, and so forth. Accordingly, as used herein, the term “hydroxyalkyl” is used to define a subset of heteroalkyl groups defined below. The term -(ar)alkyl refers to either an unsubstituted alkyl or an aralkyl group. The term (hetero)aryl or (het)aryl refers to either an aryl or a heteroaryl group.

The term “spirocycloalkyl”, as used herein, means a spirocyclic cycloalkyl group, such as, for example, spiro[3.3]heptane. The term spiroheterocycloalkyl, as used herein, means a spirocyclic heterocycloalkyl, such as, for example, 2,6-diaza spiro[3.3]heptane.

The term “acyl” as used herein denotes a group of formula —C(═O)R wherein R is hydrogen or lower alkyl as defined herein. The term or “alkylcarbonyl” as used herein denotes a group of formula C(═O)R wherein R is alkyl as defined herein. The term C₁₋₆ acyl refers to a group —C(═O)R contain 6 carbon atoms. The term “arylcarbonyl” as used herein means a group of formula C(═O)R wherein R is an aryl group; the term “benzoyl” as used herein an “arylcarbonyl” group wherein R is phenyl.

The term “ester” as used herein denotes a group of formula —C(═O)OR wherein R is lower alkyl as defined herein.

The term “alkyl” as used herein denotes an unbranched or branched chain, saturated, monovalent hydrocarbon residue containing 1 to 10 carbon atoms. The term “lower alkyl” denotes a straight or branched chain hydrocarbon residue containing 1 to 6 carbon atoms. “C₁₋₁₀ alkyl” as used herein refers to an alkyl composed of 1 to 10 carbons. Examples of alkyl groups include, but are not limited to, lower alkyl groups include methyl, ethyl, propyl, i-propyl, n-butyl, i-butyl, t-butyl or pentyl, isopentyl, neopentyl, hexyl, heptyl, and octyl.

When the term “alkyl” is used as a suffix following another term, as in “phenylalkyl,” or “hydroxyalkyl,” this is intended to refer to an alkyl group, as defined above, being substituted with one to two substituents selected from the other specifically-named group. Thus, for example, “phenylalkyl” denotes the radical R′R″—, wherein R′ is a phenyl radical, and R″ is an alkylene radical as defined herein with the understanding that the attachment point of the phenylalkyl moiety will be on the alkylene radical. Examples of arylalkyl radicals include, but are not limited to, benzyl, phenylethyl, 3-phenylpropyl. The terms “arylalkyl” or “aralkyl” are interpreted similarly except R′ is an aryl radical. The terms “(het)arylalkyl” or “(het)aralkyl” are interpreted similarly except R′ is optionally an aryl or a heteroaryl radical.

The terms “haloalkyl” or “halo-lower alkyl” or “lower haloalkyl” refers to a straight or branched chain hydrocarbon residue containing 1 to 6 carbon atoms wherein one or more carbon atoms are substituted with one or more halogen atoms.

The term “alkylene” or “alkylenyl” as used herein denotes a divalent saturated linear hydrocarbon radical of 1 to 10 carbon atoms (e.g., (CH₂)ₙ) or a branched saturated divalent hydrocarbon radical of 2 to 10 carbon atoms (e.g., —CHMe- or —CH₂CH(i-Pr)CH₂—), unless otherwise indicated. Except in the case of methylene, the open valences of an alkylene group are not attached to the same atom. Examples of alkylene radicals include, but are not limited to, methylene, ethylene, propylene, 2-methyl-propylene, 1,1-dimethyl-ethylene, butylene, 2-ethylbutylene.

The term “alkoxy” as used herein means an —O-alkyl group, wherein alkyl is as defined above such as methoxy, ethoxy, n-propyloxy, i-propyloxy, n-butyloxy, i-butyloxy, t-butyloxy, pentyloxy, hexyloxy, including their isomers. “Lower alkoxy” as used herein denotes an alkoxy group with a “lower alkyl” group as previously defined. “C₁₋₁₀ alkoxy” as used herein refers to an-O-alkyl wherein alkyl is C₁₋₁₀.

The term “PCy₃” refers to a phosphine trisubstituted with three cyclic moieties.

The terms “haloalkoxy” or “halo-lower alkoxy” or “lower haloalkoxy” refers to a lower alkoxy group, wherein one or more carbon atoms are substituted with one or more halogen atoms.

The term “hydroxyalkyl” as used herein denotes an alkyl radical as herein defined wherein one to three hydrogen atoms on different carbon atoms is/are replaced by hydroxyl groups.

The terms “alkylsulfonyl” and “arylsulfonyl” as used herein refers to a group of formula —S(═O)₂R wherein R is alkyl or aryl respectively and alkyl and aryl are as defined herein. The term “heteroalkylsulfonyl” as used herein refers herein denotes a group of formula —S(═O)₂R wherein R is “heteroalkyl” as defined herein.

The terms “alkylsulfonylamino” and “arylsulfonylamino” as used herein refers to a group of formula —NR'S(═O)₂R wherein R is alkyl or aryl respectively, R′ is hydrogen or C₁₋₃ alkyl, and alkyl and aryl are as defined herein.

The term “cycloalkyl” as used herein refers to a saturated carbocyclic ring containing 3 to 8 carbon atoms, i.e. cyclopropyl, cyclobutyl, cyclopentyl, cyclohexyl, cycloheptyl or cyclooctyl. “C₃₋₇ cycloalkyl” as used herein refers to an cycloalkyl composed of 3 to 7 carbons in the carbocyclic ring.

The term carboxy-alkyl as used herein refers to an alkyl moiety wherein one, hydrogen atom has been replaced with a carboxyl with the understanding that the point of attachment of the heteroalkyl radical is through a carbon atom. The term “carboxy” or “carboxyl” refers to a —CO₂H moiety.

The term “heteroaryl” or “heteroaromatic” as used herein means a monocyclic or bicyclic radical of 5 to 12 ring atoms having at least one aromatic or partially unsaturated ring containing four to eight atoms per ring, incorporating one or more N, O, or S heteroatoms, the remaining ring atoms being carbon, with the understanding that the attachment point of the heteroaryl radical will be on an aromatic or partially unsaturated ring. As well known to those skilled in the art, heteroaryl rings have less aromatic character than their all-carbon counter parts. Thus, for the purposes of the invention, a heteroaryl group need only have some degree of aromatic character. Examples of heteroaryl moieties include monocyclic aromatic heterocycles having 5 to 6 ring atoms and 1 to 3 heteroatoms include, but is not limited to, pyridinyl, pyrimidinyl, pyrazinyl, oxazinyl, pyrrolyl, pyrazolyl, imidazolyl, oxazolyl, 4,5-Dihydro-oxazolyl, 5,6-Dihydro-4H-[1,3]oxazolyl, isoxazole, thiazole, isothiazole, triazoline, thiadiazole and oxadiaxoline which can optionally be substituted with one or more, preferably one or two substituents selected from hydroxy, cyano, alkyl, alkoxy, thio, lower haloalkoxy, alkylthio, halo, lower haloalkyl, alkylsulfinyl, alkylsulfonyl, halogen, amino, alkylamino, dialkylamino, aminoalkyl, alkylaminoalkyl, and dialkylaminoalkyl, nitro, alkoxycarbonyl and carbamoyl, alkylcarbamoyl, dialkylcarbamoyl, arylcarbamoyl, alkylcarbonylamino and arylcarbonylamino. Examples of bicyclic moieties include, but are not limited to, quinolinyl, isoquinolinyl, benzofuryl, benzothiophenyl, benzoxazole, benzisoxazole, benzothiazole, naphthyridinyl, 5,6,7,8-Tetrahydro-[1,6]naphthyridinyl, and benzisothiazole. Bicyclic moieties can be optionally substituted on either ring, however the point of attachment is on a ring containing a heteroatom.

The term “heterocyclyl”, “heterocycloalkyl” or “heterocycle” as used herein denotes a monovalent saturated cyclic radical, consisting of one or more rings, preferably one to two rings, including spirocyclic ring systems, of three to eight atoms per ring, incorporating one or more ring heteroatoms (chosen from N, O or S(O)₀₋₂), and which can optionally be independently substituted with one or more, preferably one or two substituents selected from hydroxy, oxo, cyano, lower alkyl, lower alkoxy, lower haloalkoxy, alkylthio, halo, lower haloalkyl, hydroxyalkyl, nitro, alkoxycarbonyl, amino, alkylamino, alkylsulfonyl, arylsulfonyl, alkylaminosulfonyl, arylaminosulfonyl, alkylsulfonylamino, arylsulfonylamino, alkylaminocarbonyl, arylaminocarbonyl, alkylcarbonylamino, arylcarbonylamino, and ionic forms thereof, unless otherwise indicated. Examples of heterocyclic radicals include, but are not limited to, morpholinyl, piperazinyl, piperidinyl, azetidinyl, pyrrolidinyl, hexahydroazepinyl, oxetanyl, tetrahydrofuranyl, tetrahydrothiophenyl, oxazolidinyl, thiazolidinyl, isoxazolidinyl, tetrahydropyranyl, thiomorpholinyl, quinuclidinyl and imidazolinyl, and ionic forms thereof. Examples may also be bicyclic, such as, for example, 3,8-diaza-bicyclo[3.2.1]octane, 2,5-diaza-bicyclo[2.2.2]octane, or octahydro-pyrazino[2,1-c][1,4]oxazine.

### Inhibitors of Dengue Virus

The application provides a method for treating dengue fever comprising administering to a patient in need thereof a compound of Formula I

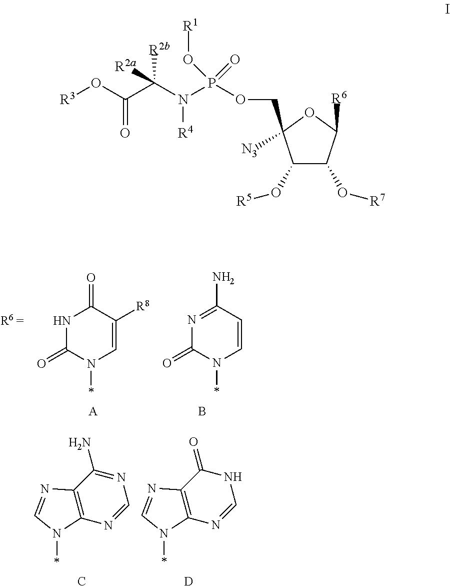

wherein:
R¹ is hydrogen, C₁₋₆haloalkyl, or aryl wherein said aryl is phenyl or naphthyl optionally substituted with one to three substituents independently selected from the group consisting of C₁₋₆ alkyl, C₂₋₆alkenyl, C₂₋₆alkynyl, C₁₋₆alkoxy, halogen, C₁₋₆haloalkyl, —N(R^{1a})₂, C₁₋₆acylamino, —NHSO₂C₁₋₆alkyl, —SO₂N(R^{1a})₂, —SO₂C₁₋₆alkyl, —COR^{1b}, nitro and cyano;
R^{1a} is independently hydrogen or C₁₋₆alkyl;
R^{1b} is —OR^{1a} or —N(R^{1a})₂;
R^{2a} and R^{2b} are (i) independently selected from the group consisting of hydrogen, C₁₋₁₀alkyl, —(CH2)ᵣN(R^{1a})₂, C₁₋₆hydroxyalkyl, —CH₂SH, —(CH₂)S(O)ₚMe, —(CH2)₃NHC(═NH)NH₂, (1H-indol-3-yl)methyl, (1H-indol-4-yl)methyl, —(CH2)ₘC(═O)R^{1b}, aryl and aryl C₁₋₃alkyl, said aryl groups optionally substituted with a group selected from the group consisting of hydroxyl, C₁₋₁₀alkyl, C₁₋₆alkoxy, halogen, nitro and cyano; (ii) R^{2a} is hydrogen and R^{2b} and R⁴ together are (CH₂)₃; (iii) R^{2a} and R^{2b} together are (CH₂)ₙ; or, (iv) R^{2a} and R^{2b} both are C₁₋₆ alkyl;
R³ is hydrogen, C₁₋₁₀ alkyl, C₁₋₁₀ haloalkyl, aryl or aryl-C₁₋₃ alkyl wherein said aryl is phenyl;
R⁴ is hydrogen, C₁₋₃ alkyl, or R^{2b} and R⁴ together are (CH₂)₃;
R⁶ is A, B, C or D wherein R⁸ is hydrogen or C₁₋₃ alkyl;
R⁵ and R⁷ is independently selected from hydrogen, C(═O)C₁₋₆alkyl, C(═O)R^{1b};
m is 0 to 3;
n is 4 or 5;
p is 0 to 2; and
r is 1 to 6;
or pharmaceutically acceptable salts thereof.

The application provides a method for treating dengue fever comprising administering to a patient in need thereof a compound of Formula Ia

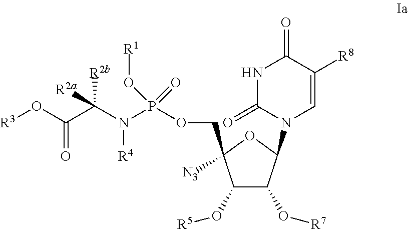

wherein:
R¹ is hydrogen, C₁₋₆haloalkyl, or aryl wherein said aryl is phenyl or naphthyl optionally substituted with one to three substituents independently selected from the group consisting of C₁₋₆ alkyl, C₂₋₆alkenyl, C₂₋₆alkynyl, C₁₋₆alkoxy, halogen, C₁₋₆haloalkyl, —N(R^{1a})₂, C₁₋₆acylamino, —NHSO₂C₁₋₆alkyl, —SO₂N(R^{1a})₂, —SO₂C₁₋₆alkyl, —COR^{1b}, nitro and cyano;
R^{1a} is independently hydrogen or C₁₋₆alkyl;
R^{1b} is —OR^{1a} or —N(R^{1a})₂;
R^{2a} and R^{2b} are (i) independently selected from the group consisting of hydrogen, C₁₋₁₀alkyl, —(CH2)ᵣN(R^{1a})₂, C₁₋₆hydroxyalkyl, —CH₂SH, —(CH₂)S(O)ₚMe, —(CH2)₃NHC(═NH)NH₂, (1H-indol-3-yl)methyl, (1H-indol-4-yl)methyl, —(CH2)ₘC(═O)R^{1b}, aryl and aryl C₁₋₃alkyl, said aryl groups optionally substituted with a group selected from the group consisting of hydroxyl, C₁₋₁₀alkyl, C₁₋₆alkoxy, halogen, nitro and cyano; (ii) R^{2a} is hydrogen and R^{2b} and R⁴ together are (CH₂)₃; (iii) R^{2a} and R^{2b} together are (CH₂)ₙ; or, (iv) R^{2a} and R^{2b} both are C₁₋₆ alkyl;
R³ is hydrogen, C₁₋₁₀ alkyl, C₁₋₁₀ haloalkyl, aryl or aryl-C₁₋₃ alkyl wherein said aryl is phenyl;
R⁴ is hydrogen, C₁₋₃ alkyl, or R^{2b} and R⁴ together are (CH₂)₃;
R⁵ and R⁷ is independently selected from hydrogen, C(═O)C₁₋₆alkyl, C(═O)R^{1b};
m is 0 to 3;
n is 4 or 5;
p is 0 to 2; and
r is 1 to 6;
or pharmaceutically acceptable salts thereof.

The application provides the above method, wherein:

R¹ is phenyl, naphthyl, or o-methoxyphenyl;
R^{2a} and R^{2b} are independently hydrogen, methyl, or benzyl;
R³ is methyl, ethyl, or benzyl;

### R⁴ is H;

R⁵ and R⁷ are both H, —C(═O)Et, or —C(═O)Bu; and

### R⁸ is H.

The application provides the above method, wherein:

R¹ is phenyl or naphthyl;
R^{2a} is hydrogen and R^{2b} is methyl;
R³ is ethyl or benzyl; and
R⁵ and R⁷ are both H or —C(═O)Et.

The application provides the above method, wherein:

R¹ is naphthyl;
R^{2a} is hydrogen and R^{2b} is methyl;
R³ is benzyl; and
R⁵ and R⁷ are both H.

In one variation, the application provides any of the above methods wherein:

R¹ is naphthyl;
R^{2a} is H and R^{2b} is benzyl;
R³ is ethyl;

### R⁴ is H;

### R⁵ is H;

### R⁶ is A;

### R⁷ is H; and

### R⁸ is H.

In another variation, the application provides any of the above methods wherein:

R¹ is naphthyl;
R^{2a} is H and R^{2b} is benzyl;
R³ is benzyl;

### R⁴ is H;

### R⁵ is H;

### R⁶ is A; and

### R⁷ is H.

In another variation, the application provides any of the above methods wherein:

R¹ is phenyl;
R^{2a} is H and R^{2b} is methyl;
R³ is benzyl;

### R⁴ is H;

### R⁵ is H;

### R⁶ is C; and

### R⁷ is H.

The application provides a method for treating dengue fever comprising administering to a patient in need thereof a compound selected from the group consisting of:
(S)-2-[[(2R,3S,4R,5R)-5-(6-Amino-purin-9-yl)-2-azido-3,4-dihydroxy-tetrahydro-furan-2-ylmethoxy]-(naphthalen-1-yloxy)-phosphorylamino]-propionic acid ethyl ester;
(S)-2-{[(2R,3S,4R,5R)-5-(6-Amino-purin-9-yl)-2-azido-3,4-dihydroxy-tetrahydro-furan-2-ylmethoxy]-phenoxy-phosphorylamino}-propionic acid benzyl ester;
(S)-2-{[(2R,3S,4R,5R)-5-(4-Amino-2-oxo-2H-pyrimidin-1-yl)-2-azido-3,4-dihydroxy-tetrahydro-furan-2-ylmethoxy]-phenoxy-phosphorylamino}-propionic acid methyl ester;
Pentanoic acid (2R,3S,4R,5R)-5-(4-amino-2-oxo-2H-pyrimidin-1-yl)-2-azido-2-[((S)-1-benzyloxycarbonyl-ethylamino)-(2-methoxy-phenoxy)-phosphoryloxymethyl]-4-pentanoyloxy-tetrahydro-furan-3-yl ester;
(S)-2-[[(2R,3S,4R,5R)-5-(4-Amino-2-oxo-2H-pyrimidin-1-yl)-2-azido-3,4-dihydroxy-tetrahydro-furan-2-ylmethoxy]-(naphthalen-1-yloxy)-phosphorylamino]-propionic acid benzyl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-propionic acid benzyl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-pentanedioic acid diethyl ester;
(S)-2-{[(2R,3S,4R,5R)-2-Azido-5-(2,4-dioxo-3,4-dihydro-2H-pyrimidin-1-yl)-3,4-bis-propionyloxy-tetrahydro-furan-2-ylmethoxy]-phenoxy-phosphorylamino}-propionic acid ethyl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-3-phenyl-propionic acid benzyl ester; and
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-3-phenyl-propionic acid ethyl ester.

The application provides any of the above methods, further comprising administering at least one other antiviral agent.

The application provides a compound selected from the group consisting of:
Pentanoic acid (2R,3S,4R,5R)-5-(4-amino-2-oxo-2H-pyrimidin-1-yl)-2-azido-2-[(((S)-1-benzyloxycarbonyl-ethylamino)-(2-methoxy-phenoxy)-phosphoryloxymethyl]-4-pentanoyloxy-tetrahydro-furan-3-yl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-pentanedioic acid diethyl ester;
(S)-2-{[(2R,3S,4R,5R)-2-Azido-5-(2,4-dioxo-3,4-dihydro-2H-pyrimidin-1-yl)-3,4-bis-propionyloxy-tetrahydro-furan-2-ylmethoxy]-phenoxy-phosphorylamino}-propionic acid ethyl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-3-phenyl-propionic acid benzyl ester; and
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-3-phenyl-propionic acid ethyl ester.

### Compounds for Method of Treating Dengue Fever

The following representative compounds of generic formula I useful for the treatment of dengue fever, as disclosed herein, are provided in the following Table I. The examples and preparations which follow are provided to enable those skilled in the art to more clearly understand and to practice the present invention. They should not be considered as limiting the scope of the invention, but merely as being illustrative and representative thereof.

In general, the nomenclature used in this Application is based on AUTONOM™ v. 4.0, a Beilstein Institute computerized system for the generation of IUPAC systematic nomenclature. If there is a discrepancy between a depicted structure and a name given that structure, the depicted structure is to be accorded more weight. In addition, if the stereochemistry of a structure or a portion of a structure is not indicated with, for example, bold or dashed lines, the structure or portion of the structure is to be interpreted as encompassing all stereoisomers of it.

TABLE I depicts examples of compounds according to generic Formulae I.

TABLE I

| Compound | Nomenclature | Structure |
|---|---|---|
| I-1 | (S)-2-[[(2R,3S,4R,5R)- 5-(6-Amino-purin-9-yl)- 2-azido-3,4-dihydroxy- tetrahydro-furan-2- ylmethoxy]- (naphthalen-1-yloxy)- phosphorylamino]- propionic acid ethyl ester | 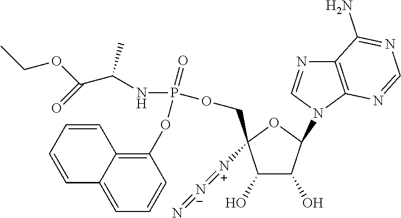 |
| I-2 | (S)-2-{[(2R,3S,4R,5R)- 5-(6-Amino-purin-9-yl)- 2-azido-3,4-dihydroxy- tetrahydro-furan-2- ylmethoxy]-phenoxy- phosphorylamino}- propionic acid benzyl ester | 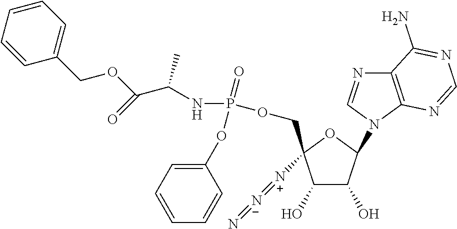 |
| I-3 | (S)-2-{[(2R,3S,4R,5R)- 5-(4-Amino-2-oxo-2H- pyrimidin-1-yl)-2- azido-3,4-dihydroxy- tetrahydro-furan-2- ylmethoxy]-phenoxy- phosphorylamino}- propionic acid methyl ester | 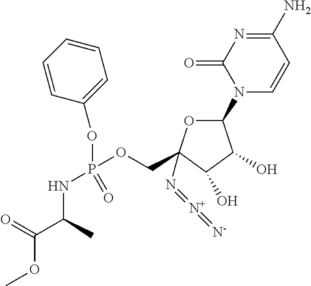 [SMILES: COC(=O)[C@H](C)NP(=O)(OC[C@@]1(N=[N+]=[N-])O[C@@H](n2ccc(N)nc2=O)[C@H](O)[C@@H]1O)Oc1ccccc1] |
| I-4 | Pentanoic acid (2R,3S,4R,5R)-5-(4- amino-2-oxo-2H- pyrimidin-1-yl)-2- azido-2-[((S)-1- benzyloxycarbonyl- ethylamino)-(2- methoxy-phenoxy)- phosphoryloxymethyl]- 4-pentanoyloxy- tetrahydro-furan-3-yl ester | 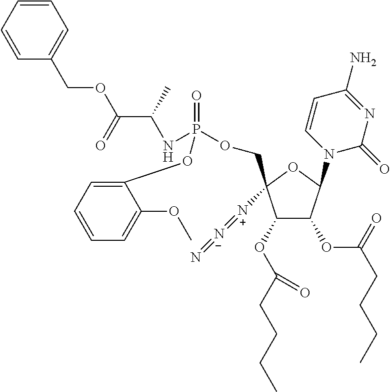 |
| I-5 | (S)-2-[[(2R,3S,4R,5R)- 5-(4-Amino-2-oxo-2H- pyrimidin-1-yl)-2- azido-3,4-dihydroxy- tetrahydro-furan-2- ylmethoxy]- (naphthalen-1-yloxy)- phosphorylamino]- propionic acid benzyl ester | 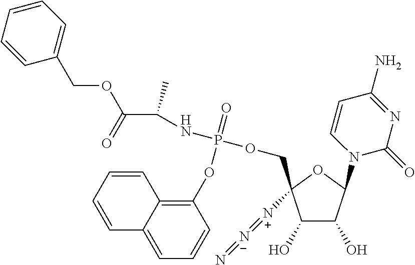 |
| I-6 | (S)-2-[[(2R,3S,4R,5R)- 2-Azido-5-(2,4-dioxo- 3,4-dihydro-2H- pyrimidin-1-yl)-3,4- dihydroxy-tetrahydro- furan-2-ylmethoxy]- (naphthalen-1-yloxy)- phosphorylamino]- propionic acid benzyl ester | 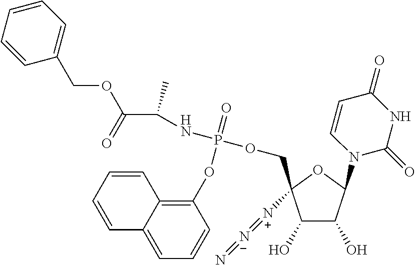 |
| I-7 | (S)-2-[[(2R,3S,4R,5R)- 2-Azido-5-(2,4-dioxo- 3,4-dihydro-2H- pyrimidin-1-yl)-3,4- dihydroxy-tetrahydro- furan-2-ylmethoxy]- (naphthalen-1-yloxy)- phosphorylamino]- pentanedioic acid diethyl ester | 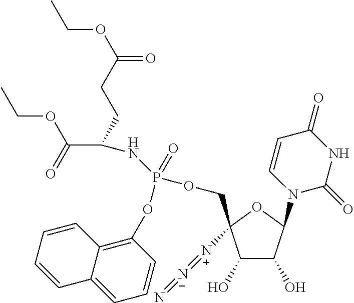 |
| I-8 | (S)-2-{[(2R,3S,4R,5R)- 2-Azido-5-(2,4-dioxo- 3,4-dihydro-2H- pyrimidin-1-yl)-3,4-bis- propionyloxy- tetrahydro-furan-2- ylmethoxy]-phenoxy- phosphorylamino}- propionic acid ethyl ester | 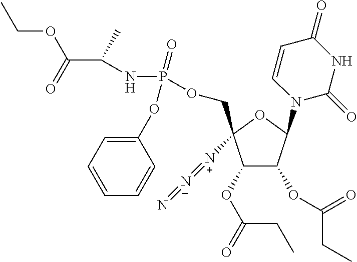 |
| I-9 | (S)-2-[[(2R,3S,4R,5R)- 2-Azido-5-(2,4-dioxo- 3,4-dihydro-2H- pyrimidin-1-yl)-3,4- dihydroxy-tetrahydro- furan-2-ylmethoxy]- (naphthalen-1-yloxy)- phosphorylamino]-3- phenyl-propionic acid benzyl ester | 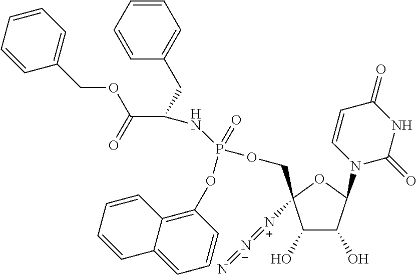 |
| I-10 | (S)-2-[[(2R,3S,4R,5R)- 2-Azido-5-(2,4-dioxo- 3,4-dihydro-2H- pyrimidin-1-yl)-3,4- dihydroxy-tetrahydro- furan-2-ylmethoxy]- (naphthalen-1-yloxy)- phosphorylamino]-3- phenyl-propionic acid ethyl ester | 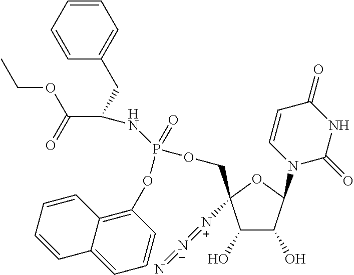 |

### Synthesis

### General Schemes

Phosphoramidate compounds of the present invention can be prepared by condensation of a 4′-azido nucleoside 4 with a suitably substituted phosphochloridate compound 3 in the presence of a strong base (Scheme 1). The nucleosides of the present invention typically contain an optionally substituted pyrimidine (R⁶=A or B) or purine (R⁶=C or D) and one or both of R⁵ and R⁷ are hydrogen or acyl or carbamoyl or alkoxycarbonyl. Examples of 4′-sazido nucleosides used to prepare compounds of the present invention can be 4′-azidoadenosine or 4′-azidouridine, which is not intended to be limiting, and the scope of the nucleosides of the present invention can be found in the claims. The condensation can be carried out on the unprotected nucleoside or, alternatively, the 2′,3′-hydroxy groups of the nucleoside can be protected as an acetonide or other diol protecting group known in the art. Deprotection of a nucleoside after the condensation is carried out utilizing standard protocols for nucleic acid chemistry.

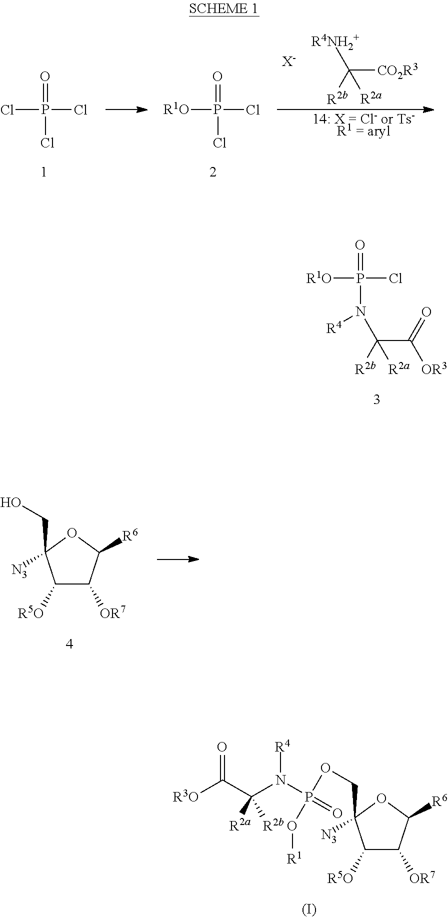

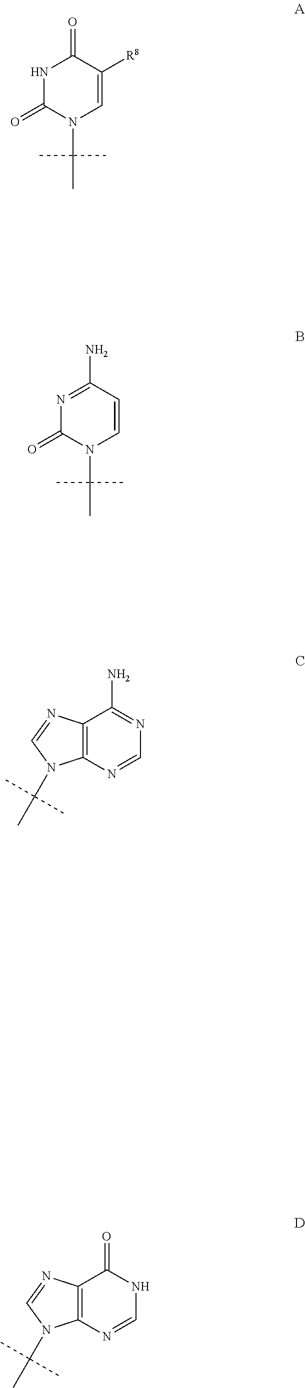

The requisite substituted phosphochloridate compounds 3 utilized to prepare compounds of the present invention are prepared by a two-step sequence comprising condensation of phosphorus oxychloride (1) with a suitably substituted phenol to afford an aryloxy phosphorodichloridates 2 which are subsequently treated with acid addition salt of an α-amino acid ester in the presence of TEA to afford an aryloxy phosphorochloridate 3 (for representative procedure see, e.g., D. Curley et al. Antiviral Res. 1990 14:345-356; C. McGuigan et al. Antiviral Res. 1992 17:311-321; McGuigan et al. Antiviral Chem. Chemother 1990 1(2):107-113).

Condensation of aryloxy phosphorochloridate 3 with a nucleoside 4 wherein R⁶ is optionally substituted uridine, cytidine, adenosine or inosine, and one or both of R⁵ and R⁷ are hydrogen or acyl or carbamoyl or alkoxycarbonyl. When R⁵ and R⁷ are both hydrogen, 2′,3′-diol can form an acetal or ketal protecting group. Treating a nucleoside with an aryloxy phosphoramidate in the presence of strong base affords the phosphoramidate derivatives of the invention (for representative procedures see, e.g. K. S. Gudmundsson, Nucleosides, Nucleotides & Nucleic Acids 2003 22(10):1953-1961). When 2′,3′-diol are protected by an acetal or ketal group, a subsequent deprotection step is required which steps are know in the art.

Compounds of formula I may exhibit tautomerism. Tautomeric compounds can exist as two or more interconvertable species. Prototropic tautomers result from the migration of a covalently bonded hydrogen atom between two atoms. Tautomers generally exist in equilibrium and attempts to isolate an individual tautomers usually produce a mixture whose chemical and physical properties are consistent with a mixture of compounds. The position of the equilibrium is dependent on chemical features within the molecule. For example, in many aliphatic aldehydes and ketones, such as acetaldehyde, the keto form predominates while; in phenols, the enol form predominates. Common prototropic tautomers include keto/enol (—C(═O)—CH—⇄—C(—OH)═CH—), amide/imidic acid (—C(═O)—NH—⇄—C(—OH)═N—) and amidine (—C(═NR)—NH—⇄—C(—NHR)═N—) tautomers. The latter two are particularly common in heteroaryl and heterocyclic rings and the present invention encompasses all tautomeric forms of the compounds.

The term “amino acid” as used herein refers to naturally occurring α amino carboxylic acids, as well as to optical isomers (enantiomers and diastereomers), synthetic analogs and derivatives thereof α-Amino acids comprise a carbon atom bonded to a carboxyl group, an amino group, a hydrogen atom and a unique “side chain” group. The term “naturally occurring amino acids” means the L-isomers of the naturally occurring amino acids. The naturally occurring amino acids are glycine, alanine, valine, leucine, isoleucine, serine, methionine, threonine, phenylalanine, tyrosine, tryptophan, cysteine, proline, histidine, aspartic acid, asparagine, glutamic acid, glutamine, γ-carboxyglutamic acid, arginine, ornithine and lysine. The side chains of naturally occurring amino acids include: hydrogen, methyl, iso-propyl, iso-butyl, sec-butyl, —CH₂OH, —CH(OH)CH₃, —CH₂SH, —CH₂CH₂SMe, —(CH₂)ₚCOR wherein R is —OH or —NH₂ and p is 1 or 2, —(CH₂)_{q}—NH₂ where q is 3 or 4, —(CH₂)₃—NHC(═NH)NH₂, —CH₂C₆H₅, —CH₂-p-C₆H₄—OH, (3-indolinyl)methylene, (4-imidazolyl)methylene.

Compounds of the present invention may have asymmetric centers located on the side chain of a carboxylic ester, amide or carbonate moiety that produce diastereomers when linked to the nucleoside. All stereoisomers of a side chain of compounds of the instant invention are contemplated, either in admixture or in pure or substantially pure form. The definition of the compounds according to the invention embraces all both isolated optical isomers enantiomers and their mixtures including the racemic form. The pure optical isomer can be prepared by stereospecific synthesis from α-D-ribose or the racemic form can be resolved by physical methods, such as, for example, fractional crystallization, separation or crystallization of diastereomeric derivatives or separation by chiral column chromatography. The individual optical isomers can be obtained from the racemates by conventional methods, such as, for example, salt formation with an optically active acid followed by crystallization.

### Pharmaceutical Compositions and Administration

Pharmaceutical compositions of the subject Compounds for administration via several routes were prepared as described in this Example.

Composition for Oral Administration (A)

| Ingredient | % wt./wt. |
|---|---|
| Active ingredient | 20.0% |
| Lactose | 79.5% |
| Magnesium stearate | 0.5% |

The ingredients are mixed and dispensed into capsules containing about 100 mg each; one capsule would approximate a total daily dosage.

Composition for Oral Administration (B)

| Ingredient | % wt./wt. |
|---|---|
| Active ingredient | 20.0% |
| Magnesium stearate | 0.5% |
| Crosscarmellose | 2.0% |
| sodium | |
| Lactose | 76.5% |
| PVP | 1.0% |
| (polyvinylpyrrolidine) | |

The ingredients are combined and granulated using a solvent such as methanol. The formulation is then dried and formed into tablets (containing about 20 mg of active compound) with an appropriate tablet machine.

Composition for Oral Administration (C)

| Ingredient | % wt./wt. | |
|---|---|---|
| Active compound | 1.0 | g |
| Fumaric acid | 0.5 | g |
| Sodium chloride | 2.0 | g |
| Methyl paraben | 0.15 | g |
| Propyl paraben | 0.05 | g |
| Granulated sugar | 25.5 | g |
| Sorbitol (70% solution) | 12.85 | g |
| Veegum K (Vanderbilt Co.) | 1.0 | g |
| Flavoring | 0.035 | ml |
| Colorings | 0.5 | mg |

| | |
|---|---|
| Distilled water | q.s. to 100 ml |

The ingredients are mixed to form a suspension for oral administration.

Parenteral Formulation (D)

| Ingredient | % wt./wt. | |
|---|---|---|
| Active ingredient | 0.25 | g |
| Sodium Chloride | qs to make isotonic | |
| Water for injection to | 100 | ml |

The active ingredient is dissolved in a portion of the water for injection. A sufficient quantity of sodium chloride is then added with stirring to make the solution isotonic. The solution is made up to weight with the remainder of the water for injection, filtered through a 0.2 micron membrane filter and packaged under sterile conditions.

### Dosage and Administration:

The compounds of the present invention may be formulated in a wide variety of oral administration dosage forms and carriers. Oral administration can be in the form of tablets, coated tablets, dragées, hard and soft gelatin capsules, solutions, emulsions, syrups, or suspensions. Compounds of the present invention are efficacious when administered by other routes of administration including continuous (intravenous drip) topical parenteral, intramuscular, intravenous, subcutaneous, transdermal (which may include a penetration enhancement agent), buccal, nasal, inhalation and suppository administration, among other routes of administration. The preferred manner of administration is generally oral using a convenient daily dosing regimen which can be adjusted according to the degree of affliction and the patient's response to the active ingredient.

A compound or compounds of the present invention, as well as their pharmaceutically useable salts, together with one or more conventional excipients, carriers, or diluents, may be placed into the form of pharmaceutical compositions and unit dosages. The pharmaceutical compositions and unit dosage forms may be comprised of conventional ingredients in conventional proportions, with or without additional active compounds or principles, and the unit dosage forms may contain any suitable effective amount of the active ingredient commensurate with the intended daily dosage range to be employed. The pharmaceutical compositions may be employed as solids, such as tablets or filled capsules, semisolids, powders, sustained release formulations, or liquids such as solutions, suspensions, emulsions, elixirs, or filled capsules for oral use; or in the form of suppositories for rectal or vaginal administration; or in the form of sterile injectable solutions for parenteral use. A typical preparation will contain from about 5% to about 95% active compound or compounds (w/w). The term “preparation” or “dosage form” is intended to include both solid and liquid formulations of the active compound and one skilled in the art will appreciate that an active ingredient can exist in different preparations depending on the target organ or tissue and on the desired dose and pharmacokinetic parameters.

The term “excipient” as used herein refers to a compound that is useful in preparing a pharmaceutical composition, generally safe, non-toxic and neither biologically nor otherwise undesirable, and includes excipients that are acceptable for veterinary use as well as human pharmaceutical use. The compounds of this invention can be administered alone but will generally be administered in admixture with one or more suitable pharmaceutical excipients, diluents or carriers selected with regard to the intended route of administration and standard pharmaceutical practice.

“Pharmaceutically acceptable” means that which is useful in preparing a pharmaceutical composition that is generally safe, non-toxic, and neither biologically nor otherwise undesirable and includes that which is acceptable for veterinary as well as human pharmaceutical use.

A “pharmaceutically acceptable salt” form of an active ingredient may also initially confer a desirable pharmacokinetic property on the active ingredient which were absent in the non-salt form, and may even positively affect the pharmacodynamics of the active ingredient with respect to its therapeutic activity in the body. The phrase “pharmaceutically acceptable salt” of a compound means a salt that is pharmaceutically acceptable and that possesses the desired pharmacological activity of the parent compound. Such salts include: (1) acid addition salts, formed with inorganic acids such as hydrochloric acid, hydrobromic acid, sulfuric acid, nitric acid, phosphoric acid, and the like; or formed with organic acids such as acetic acid, propionic acid, hexanoic acid, cyclopentanepropionic acid, glycolic acid, pyruvic acid, lactic acid, malonic acid, succinic acid, malic acid, maleic acid, fumaric acid, tartaric acid, citric acid, benzoic acid, 3-(4-hydroxybenzoyl)benzoic acid, cinnamic acid, mandelic acid, methanesulfonic acid, ethanesulfonic acid, 1,2-ethane-disulfonic acid, 2-hydroxyethanesulfonic acid, benzenesulfonic acid, 4-chlorobenzenesulfonic acid, 2-naphthalenesulfonic acid, 4-toluenesulfonic acid, camphorsulfonic acid, 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like; or (2) salts formed when an acidic proton present in the parent compound either is replaced by a metal ion, e.g., an alkali metal ion, an alkaline earth ion, or an aluminum ion; or coordinates with an organic base such as ethanolamine, diethanolamine, triethanolamine, tromethamine, N-methylglucamine, and the like.

Solid form preparations include powders, tablets, pills, capsules, cachets, suppositories, and dispersible granules. A solid carrier may be one or more substances which may also act as diluents, flavoring agents, solubilizers, lubricants, suspending agents, binders, preservatives, tablet disintegrating agents, or an encapsulating material. In powders, the carrier generally is a finely divided solid which is a mixture with the finely divided active component. In tablets, the active component generally is mixed with the carrier having the necessary binding capacity in suitable proportions and compacted in the shape and size desired. Suitable carriers include but are not limited to magnesium carbonate, magnesium stearate, talc, sugar, lactose, pectin, dextrin, starch, gelatin, tragacanth, methylcellulose, sodium carboxymethylcellulose, a low melting wax, cocoa butter, and the like. Solid form preparations may contain, in addition to the active component, colorants, flavors, stabilizers, buffers, artificial and natural sweeteners, dispersants, thickeners, solubilizing agents, and the like.

Liquid formulations also are suitable for oral administration include liquid formulation including emulsions, syrups, elixirs, aqueous solutions, aqueous suspensions. These include solid form preparations which are intended to be converted to liquid form preparations shortly before use. Emulsions may be prepared in solutions, for example, in aqueous propylene glycol solutions or may contain emulsifying agents such as lecithin, sorbitan monooleate, or acacia. Aqueous solutions can be prepared by dissolving the active component in water and adding suitable colorants, flavors, stabilizing, and thickening agents. Aqueous suspensions can be prepared by dispersing the finely divided active component in water with viscous material, such as natural or synthetic gums, resins, methylcellulose, sodium carboxymethylcellulose, and other well known suspending agents.

The compounds of the present invention may be formulated for parenteral administration (e.g., by injection, for example bolus injection or continuous infusion) and may be presented in unit dose form in ampoules, pre-filled syringes, small volume infusion or in multidose containers with an added preservative. The compositions may take such forms as suspensions, solutions, or emulsions in oily or aqueous vehicles, for example solutions in aqueous polyethylene glycol. Examples of oily or nonaqueous carriers, diluents, solvents or vehicles include propylene glycol, polyethylene glycol, vegetable oils (e.g., olive oil), and injectable organic esters (e.g., ethyl oleate), and may contain formulatory agents such as preserving, wetting, emulsifying or suspending, stabilizing and/or dispersing agents. Alternatively, the active ingredient may be in powder form, obtained by aseptic isolation of sterile solid or by lyophilisation from solution for constitution before use with a suitable vehicle, e.g., sterile, pyrogen-free water.

The compounds of the present invention may be formulated for topical administration to the epidermis as ointments, creams or lotions, or as a transdermal patch. Ointments and creams may, for example, be formulated with an aqueous or oily base with the addition of suitable thickening and/or gelling agents. Lotions may be formulated with an aqueous or oily base and will in general also containing one or more emulsifying agents, stabilizing agents, dispersing agents, suspending agents, thickening agents, or coloring agents. Formulations suitable for topical administration in the mouth include lozenges comprising active agents in a flavored base, usually sucrose and acacia or tragacanth; pastilles comprising the active ingredient in an inert base such as gelatin and glycerin or sucrose and acacia; and mouthwashes comprising the active ingredient in a suitable liquid carrier.

The compounds of the present invention may be formulated for administration as suppositories. A low melting wax, such as a mixture of fatty acid glycerides or cocoa butter is first melted and the active component is dispersed homogeneously, for example, by stirring. The molten homogeneous mixture is then poured into convenient sized molds, allowed to cool, and to solidify.

The compounds of the present invention may be formulated for vaginal administration. Pessaries, tampons, creams, gels, pastes, foams or sprays containing in addition to the active ingredient such carriers as are known in the art to be appropriate.

The compounds of the present invention may be formulated for nasal administration. The solutions or suspensions are applied directly to the nasal cavity by conventional means, for example, with a dropper, pipette or spray. The formulations may be provided in a single or multidose form. In the latter case of a dropper or pipette, this may be achieved by the patient administering an appropriate, predetermined volume of the solution or suspension. In the case of a spray, this may be achieved for example by means of a metering atomizing spray pump.

The compounds of the present invention may be formulated for aerosol administration, particularly to the respiratory tract and including intranasal administration. The compound will generally have a small particle size for example of the order of five (5) microns or less. Such a particle size may be obtained by means known in the art, for example by micronization. The active ingredient is provided in a pressurized pack with a suitable propellant such as a chlorofluorocarbon (CFC), for example, dichlorodifluoromethane, trichlorofluoromethane, or dichlorotetrafluoroethane, or carbon dioxide or other suitable gas. The aerosol may conveniently also contain a surfactant such as lecithin. The dose of drug may be controlled by a metered valve. Alternatively the active ingredients may be provided in a form of a dry powder, for example a powder mix of the compound in a suitable powder base such as lactose, starch, starch derivatives such as hydroxypropylmethyl cellulose and polyvinylpyrrolidine (PVP). The powder carrier will form a gel in the nasal cavity. The powder composition may be presented in unit dose form for example in capsules or cartridges of e.g., gelatin or blister packs from which the powder may be administered by means of an inhaler.

When desired, formulations can be prepared with enteric coatings adapted for sustained or controlled release administration of the active ingredient. For example, the compounds of the present invention can be formulated in transdermal or subcutaneous drug delivery devices. These delivery systems are advantageous when sustained release of the compound is necessary and when patient compliance with a treatment regimen is crucial. Compounds in transdermal delivery systems are frequently attached to an skin-adhesive solid support. The compound of interest can also be combined with a penetration enhancer, e.g., Azone (1-dodecylaza-cycloheptan-2-one). Sustained release delivery systems are inserted subcutaneously into to the subdermal layer by surgery or injection. The subdermal implants encapsulate the compound in a lipid soluble membrane, e.g., silicone rubber, or a biodegradable polymer, e.g., polylactic acid.

Suitable formulations along with pharmaceutical carriers, diluents and excipients are described in Remington: The Science and Practice of Pharmacy 1995, edited by E. W. Martin, Mack Publishing Company, 19th edition, Easton, Pa. A skilled formulation scientist may modify the formulations within the teachings of the specification to provide numerous formulations for a particular route of administration without rendering the compositions of the present invention unstable or compromising their therapeutic activity.

The modification of the present compounds to render them more soluble in water or other vehicle, for example, may be easily accomplished by minor modifications (salt formulation, esterification, etc.), which are well within the ordinary skill in the art. It is also well within the ordinary skill of the art to modify the route of administration and dosage regimen of a particular compound in order to manage the pharmacokinetics of the present compounds for maximum beneficial effect in patients.

The term “therapeutically effective amount” as used herein means an amount required to reduce symptoms of the disease in an individual. The dose will be adjusted to the individual requirements in each particular case. That dosage can vary within wide limits depending upon numerous factors such as the severity of the disease to be treated, the age and general health condition of the patient, other medicaments with which the patient is being treated, the route and form of administration and the preferences and experience of the medical practitioner involved. For oral administration, a daily dosage of between about 0.01 and about 1000 mg/kg body weight per day should be appropriate in monotherapy and/or in combination therapy. A preferred daily dosage is between about 0.1 and about 500 mg/kg body weight, more preferred 0.1 and about 100 mg/kg body weight and most preferred 1.0 and about 10 mg/kg body weight per day. Thus, for administration to a 70 kg person, the dosage range would be about 7 mg to 0.7 g per day. The daily dosage can be administered as a single dosage or in divided dosages, typically between 1 and 5 dosages per day. Generally, treatment is initiated with smaller dosages which are less than the optimum dose of the compound. Thereafter, the dosage is increased by small increments until the optimum effect for the individual patient is reached. One of ordinary skill in treating diseases described herein will be able, without undue experimentation and in reliance on personal knowledge, experience and the disclosures of this application, to ascertain a therapeutically effective amount of the compounds of the present invention for a given disease and patient.

The pharmaceutical preparations are preferably in unit dosage forms. In such form, the preparation is subdivided into unit doses containing appropriate quantities of the active component. The unit dosage form can be a packaged preparation, the package containing discrete quantities of preparation, such as packeted tablets, capsules, and powders in vials or ampoules. Also, the unit dosage form can be a capsule, tablet, cachet, or lozenge itself, or it can be the appropriate number of any of these in packaged form.

### Indications and Method of Treatment

The compounds of the invention and their isomeric forms and pharmaceutically acceptable salts thereof are useful in treating and preventing dengue virus infection.

The application provides a method for treating a dengue virus infection comprising administering to a patient in need thereof a therapeutically effective amount of a compound of Formula I.

The application provides a method for inhibiting replication of dengue virus in a cell comprising administering a compound of Formula I.

### EXAMPLES

### Abbreviations

Commonly used abbreviations include: acetyl (Ac), azo-bis-isobutyrylnitrile (AIBN), atmospheres (Atm), 9-borabicyclo[3.3.1]nonane (9-BBN or BBN), 2,2′-bis(diphenylphosphino)-1,1′-binaphthyl (BINAP), tert-butoxycarbonyl (Boc), di-tert-butyl pyrocarbonate or boc anhydride (BOC₂O), benzyl (Bn), butyl (Bu), Chemical Abstracts Registration Number (CASRN), benzyloxycarbonyl (CBZ or Z), carbonyl diimidazole (CDI), 1,4-diazabicyclo[2.2.2]octane (DABCO), diethylaminosulfur trifluoride (DAST), dibenzylideneacetone (dba), 1,5-diazabicyclo[4.3.0]non-5-ene (DBN), 1,8-diazabicyclo[5.4.0]undec-7-ene (DBU), N,N′-dicyclohexylcarbodiimide (DCC), 1,2-dichloroethane (DCE), dichloromethane (DCM), 2,3-Dichloro-5,6-dicyano-1,4-benzoquinone (DDQ), diethyl azodicarboxylate (DEAD), di-iso-propylazodicarboxylate (DIAD), di-iso-butylaluminumhydride (DIBAL or DIBAL-H), di-iso-propylethylamine (DIPEA), N,N-dimethyl acetamide (DMA), 4-N,N-dimethylaminopyridine (DMAP), N,N-dimethylformamide (DMF), dimethyl sulfoxide (DMSO), 1,1′-bis-(diphenylphosphino)ethane (dppe), 1,1′-bis-(diphenylphosphino)ferrocene (dppf), 1-(3-dimethylaminopropyl)-3-ethylcarbodiimide hydrochloride (EDCI), 2-ethoxy-1-ethoxycarbonyl-1,2-dihydroquinoline (EEDQ), ethyl (Et), ethyl acetate (EtOAc), ethanol (EtOH), 2-ethoxy-2H-quinoline-1-carboxylic acid ethyl ester (EEDQ), diethyl ether (Et₂O), ethyl isopropyl ether (EtOiPr), O-(7-azabenzotriazole-1-yl)-N, N,N′N′-tetramethyluronium hexafluorophosphate acetic acid (HATU), acetic acid (HOAc), 1-N-hydroxybenzotriazole (HOBt), high pressure liquid chromatography (HPLC), iso-propanol (IPA), isopropylmagnesium chloride (iPrMgCl), hexamethyl disilazane (HMDS), liquid chromatography mass spectrometry (LCMS), lithium hexamethyl disilazane (LiHMDS), meta-chloroperoxybenzoic acid (m-CPBA), methanol (MeOH), melting point (mp), MeSO₂— (mesyl or Ms), methyl (Me), acetonitrile (MeCN), m-chloroperbenzoic acid (MCPBA), mass spectrum (ms), methyl t-butyl ether (MTBE), methyl tetrahydrofuran (MeTHF), N-bromosuccinimide (NBS), n-Butyllithium (nBuLi), N-carboxyanhydride (NCA), N-chlorosuccinimide (NCS), N-methylmorpholine (NMM), N-methylpyrrolidone (NMP), pyridinium chlorochromate (PCC), Dichloro-((bis-diphenylphosphino)ferrocenyl) palladium(II) (Pd(dppf)Cl₂), palladium(II) acetate (Pd(OAc)₂), tris(dibenzylideneacetone)dipalladium(0) (Pd₂(dba)₃), pyridinium dichromate (PDC), phenyl (Ph), propyl (Pr), iso-propyl (i-Pr), pounds per square inch (psi), pyridine (pyr), 1,2,3,4,5-Pentaphenyl-1′-(di-tert-butylphosphino)ferrocene (Q-Phos), room temperature (ambient temperature, rt or RT), sec-Butyllithium (sBuLi), tert-butyldimethylsilyl or t-BuMe₂Si (TBDMS), tetra-n-butylammonium fluoride (TBAF), triethylamine (TEA or Et₃N), 2,2,6,6-tetramethylpiperidine 1-oxyl (TEMPO), triflate or CF₃SO₂— (Tf), trifluoroacetic acid (TFA), 1,1′-bis-2,2,6,6-tetramethylheptane-2,6-dione (TMHD), O-benzotriazol-1-yl-N,N,N′,N′-tetramethyluronium tetrafluoroborate (TBTU), thin layer chromatography (TLC), tetrahydrofuran (THF), trimethylsilyl or Me₃Si (TMS), p-toluenesulfonic acid monohydrate (TsOH or pTsOH), 4-Me-C₆H₄SO₂— or tosyl (Ts), and N-urethane-N-carboxyanhydride (UNCA). Conventional nomenclature including the prefixes normal (n), iso (i-), secondary (sec-), tertiary (tert-) and neo have their customary meaning when used with an alkyl moiety. (J. Rigaudy and D. P. Klesney, Nomenclature in Organic Chemistry, IUPAC 1979 Pergamon Press, Oxford.).

### General Conditions

Compounds of the invention can be made by a variety of methods depicted in the illustrative synthetic reactions described below in the Examples section. U.S. Pat. No. 7,608,599 discloses the preparation of antiviral nucleoside phosphoramidates and is herein incorporated by reference in its entirety.

The starting materials and reagents used in preparing these compounds generally are either available from commercial suppliers, such as Aldrich Chemical Co., or are prepared by methods known to those skilled in the art following procedures set forth in references such as Fieser and Fieser's Reagents for Organic Synthesis; Wiley & Sons: New York, 1991, Volumes 1-15; Rodd's Chemistry of Carbon Compounds, Elsevier Science Publishers, 1989, Volumes 1-5 and Supplementals; and Organic Reactions, Wiley & Sons: New York, 1991, Volumes 1-40. It should be appreciated that the synthetic reaction schemes shown in the Examples section are merely illustrative of some methods by which the compounds of the invention can be synthesized, and various modifications to these synthetic reaction schemes can be made and will be suggested to one skilled in the art having referred to the disclosure contained in this application.

The starting materials and the intermediates of the synthetic reaction schemes can be isolated and purified if desired using conventional techniques, including but not limited to, filtration, distillation, crystallization, chromatography, and the like. Such materials can be characterized using conventional means, including physical constants and spectral data.

Unless specified to the contrary, the reactions described herein are typically conducted under an inert atmosphere at atmospheric pressure at a reaction temperature range of from about −78° C. to about 150° C., often from about 0° C. to about 125° C., and more often and conveniently at about room (or ambient) temperature, e.g., about 20° C.

Various substituents on the compounds of the invention can be present in the starting compounds, added to any one of the intermediates or added after formation of the final products by known methods of substitution or conversion reactions. If the substituents themselves are reactive, then the substituents can themselves be protected according to the techniques known in the art. A variety of protecting groups are known in the art, and can be employed. Examples of many of the possible groups can be found in “Protective Groups in Organic Synthesis” by Green et al., John Wiley and Sons, 1999. For example, nitro groups can be added by nitration and the nitro group can be converted to other groups, such as amino by reduction, and halogen by diazotization of the amino group and replacement of the diazo group with halogen. Acyl groups can be added by Friedel-Crafts acylation. The acyl groups can then be transformed to the corresponding alkyl groups by various methods, including the Wolff-Kishner reduction and Clemmenson reduction. Amino groups can be alkylated to form mono- and di-alkylamino groups; and mercapto and hydroxy groups can be alkylated to form corresponding ethers. Primary alcohols can be oxidized by oxidizing agents known in the art to form carboxylic acids or aldehydes, and secondary alcohols can be oxidized to form ketones. Thus, substitution or alteration reactions can be employed to provide a variety of substituents throughout the molecule of the starting material, intermediates, or the final product, including isolated products.

### General Methodology

TLC was carried out on precoated, aluminum backed plates (60 F-54, 0.2 mm thickness; supplied by E. Merck AG, Darmstad, Germany) developed by ascending method. After solvent evaporation, compounds were detected by irradiation with an UV lamp at 254 nm or 366 nm observation of quenching of the fluorescence. Chromatography columns were slurry packed in the appropriate eluent under pressure, with silica gel, 60 A, 40-60 μm, Phase Sep, UK). Samples were applied as a concentrated solution in the same eluent, or pre-adsorbed on silica gel. ¹H and ¹³C NMR spectra were recorded on a Bruker Advance DPX300 spectrometer (300 MHz and 75 MHz respectively) and autocalibrated to the deuterated solvent reference peak. All ¹³C NMR were proton decoupled. The following abbreviations are used in the assignment of NMR signals: s (singlet), d (doublet), t (triplet), qu (quartet), q (quintet), m (multiplet), bs (broad signal), dd (double doublet), dt (double triplet). Low-resolution mass spectra were run on a VG Platform II Fisons instrument (atmospheric pressure ionization, electrospray mass spectrometry) in either negative or positive mode.

The solvents used were anhydrous and used as purchased from Aldrich. All glassware was oven dried at 130° C. for several hours and allowed to cool under a stream of dry nitrogen.

### Preparative Examples

### Example 1

### 4′-Azidoadenosine 5′-O-[Phenyhbenzyloxy-L-alaninyl)]Phosphate

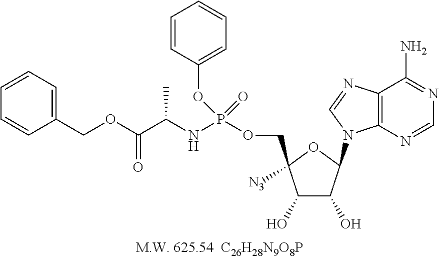 [SMILES: C[C@H](NP(=O)(OC[C@@]1(N=[N+]=[N-])O[C@@H](n2cnc3c(N)ncnc32)[C@H](O)[C@@H]1O)Oc1ccccc1)C(=O)OCc1ccccc1]

The preparation of the titled compound (I-2) has been disclosed by McGuigan, Christopher, et al in Journal of Medicinal Chemistry (2007), 50(22), 5463-5470.

HRMS (E/I) m/e 648.1696 (MNa⁺). Accurate mass: C₂₆H₂₈N₉O₈NaP requires 648.1696.

### Example 2

### 4′-Azidoadenosine 5′-O-[naphtha-1-yl(benzyloxy-L-phenylalaninyl)]Phosphate

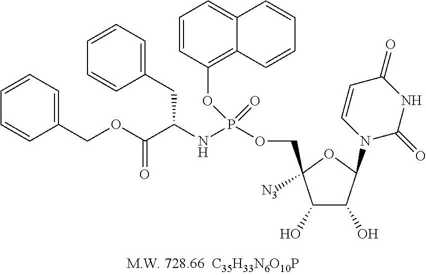 [SMILES: [N-]=[N+]=N[C@]1(COP(=O)(N[C@@H](Cc2ccccc2)C(=O)OCc2ccccc2)Oc2cccc3ccccc23)O[C@@H](n2ccc(=O)[nH]c2=O)[C@H](O)[C@@H]1O]

The titled compound (I-9) was prepared in a similar manner to the methods described by McGuigan, Christopher et al in Journal of Medicinal Chemistry (2007), 50(8), 1840-1849.

### Example 3

### 4′-Azidoadenosine 5′-O-[naphtha-1-yl(ethyloxy-L-phenylalaninyl)]Phosphate

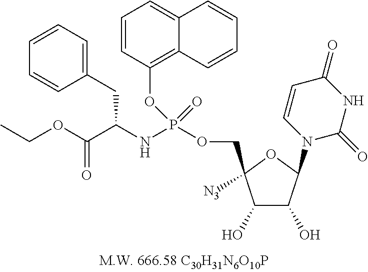 [SMILES: CCOC(=O)[C@H](Cc1ccccc1)NP(=O)(OC[C@@]1(N=[N+]=[N-])O[C@@H](n2ccc(=O)[nH]c2=O)[C@H](O)[C@@H]1O)Oc1cccc2ccccc12]

The titled compound (I-10) was prepared in a similar manner to the methods described by McGuigan, Christopher et al in Journal of Medicinal Chemistry (2007), 50(8), 1840-1849.

### Biological Examples

### Huh7 Cells Antiviral Assay

The human hepatoma cell line Huh-7 (Mainz University, Germany), were cultured in DMEM without phenol-red (Cellgro Mediatech, Cat #10-013-CV containing 4.5 g/l glucose, L-glutamine & sodium pyruvate). The medium was further supplemented with 10% (v/v) FBS (ATLAS Cat # F-0500-A, lot#850114A) and 1% (vlv) penicillin/streptomycin (Cellgro Mediatech #30-022-CI). Cells were maintained at 37° C. in a humidified 5% CO₂ atmosphere. Dengue virus representative strains of the four serotypes DENV-1 (Th-Sman), DENV-2 (Th-36), DENV-3 (H-87) and DENV-4 (H-241) were all obtained from the ATCC (Manassas, Va.). Virus titers were measured on BHK-21 cells, using a standard plaque assay procedure. For the determination of EC₅₀ of nucleoside in the antiviral assay, Huh-7 cells were plated in white 96-well plates in MEM media supplemented with 10% FBS and 1% penicillin/streptomycin. After incubation for 24 h, cells were infected at a multiplicity of infection (MOI) of 0.5 for 2 h at 37° C. Ten three-fold dilutions of compounds were prepared in the same media supplemented with 1% DMSO. After the 2 h adsorption phase, virus was aspirated off and diluted compound was added to four wells each. Huh-7 cells were plated as described above and exposed to the same concentration range of compounds. Untreated cells were carried along as a control. After a 3-day incubation at 37° C., the cell viability was determined using Cell-titer Glo™ reagent (Promega, Madison, Wis.) that was added to each well and incubated for 5 min. Plates were analyzed using a Thermo Luminoskan plate reader (Waltham, Mass.).

### Dendritic Cells Infection Assay

Cryopreserved human immature monocyte derived Dendritic Cells (iDC) from individual donors were obtained from Stemcell Technologies (CAT# PB-DC001F). iDC were counted and incubated at a concentration of 15,000 cells/well (96 well plate) in RPMI 1640 media containing 10% (v/v) fetal bovine serum (FBS), 1% (v/v) penicillin/streptomycin (Invitrogen™) for 48 h at 37° C. in a 90% humidified, 5% CO₂ atmosphere prior to the start of the experiment.

In a 96 well flat-bottom plate, 15000 iDC from individual donors were infected with dengue virus at a multiplicity of infection (MOI) of 2 in a volume of 50 μl for 2 h. After Infection iDCs were washed and cultured in complete RPMI media in the presence of serially diluted compounds. Each virus/drug combination was tested either in duplicate or triplicate (Depending on the availability of iDC from individual donors). Plates were incubated for 24 h at 37° C. in a 90% humidified, 5% CO₂ atmosphere. After 24 h cells were washed and cellular RNA were isolated. Viral RNA and endogenous 18S rRNA control (Applied Bio Systems) was quantified by a real time PCR assay. The viability of mock-infected and infected iDC were monitored at described time points using a CellTiter Glo® (promega) assay according to manufacturer's recommendation.

Cellular RNA was isolated by PerfectPure™ RNA 96 cell kit (5 PRIME) according to manufacturer's recommendation. Transcriptor First Strand cDNA Synthesis Kit (Roche) was used to generate cDNA using random hexamer primers. 5 μl of generated cDNA was subjected to a real time PCR assay (Roche) using the primers targeting dengue 3′ UTR and the following primers: dengue reverse (Common to all the serotypes): 5′-GATCTCTGGTCTTTCCCAGCGTCAA-3′, dengue forward serotype 1: 5′-GAGCCCCGTCCAAGGACGTAAAATGAA-3′, dengue forward serotypes 2 or 3: 5′-GAGCCCCGTCCAAGGACGTTAAAAGAA-3′, dengue′ forward serotype 4: 5′-TATTGAAGTCAGGCCACTTGTGCC-3′ and dengue probe (Common to all the serotypes): 5′-/56 FAM/AAGGACTAGAGGTTAGAGGAGACCCCCCGC/3BHQ1/-3′. All the primers were obtained from Integrated DNA Technologies. Taqman was performed in duplicate. Percentage of inhibition was obtained using the following calculations. First ΔCt was calculated by subtracting the 18S rRNA CT value from the dengue RNA CT value. ΔCts from duplicate taqman assay were averaged. Then ΔΔCt was obtained by subtracting the average ΔCt of a non treated sample from the treated sample average ΔCt. Relative quantification was calculated using the following formula. Relative quantification=2^{-averageΔΔCT}. The 50% inhibitory concentrations (IC₅₀) were calculated using the sigmoidal dose-response model in Microsoft XLfit.

Representative assay data can be found in Table II below:

TABLE II

| | IC50 (H241: dengue virus serotype 4) |
|---|---|
| | (In human primary |
| Compound # | Dendritic cells) μM |
| I-2 | 1.9 |
| I-9 | 16.0 |
| I-10 | 15.0 |

The foregoing invention has been described in some detail by way of illustration and example, for purposes of clarity and understanding. It will be obvious to one of skill in the art that changes and modifications may be practiced within the scope of the appended claims. Therefore, it is to be understood that the above description is intended to be illustrative and not restrictive. The scope of the invention should, therefore, be determined not with reference to the above description, but should instead be determined with reference to the following appended claims, along with the full scope of equivalents to which such claims are entitled.

All patents, patent applications and publications cited in this application are hereby incorporated by reference in their entirety for all purposes to the same extent as if each individual patent, patent application or publication were so individually denoted.

We claim:

1. A method for treating dengue fever comprising administering to a patient in need thereof a compound of Formula I 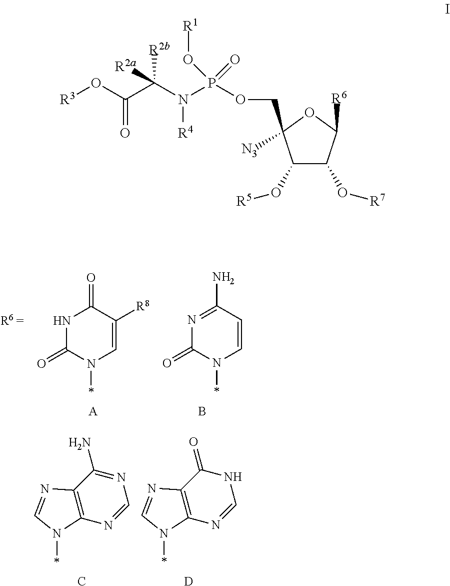
wherein:
R¹ is hydrogen, C₁₋₆haloalkyl, or aryl wherein said aryl is phenyl or naphthyl optionally substituted with one to three substituents independently selected from the group consisting of C₁₋₆ alkyl, C₂₋₆ alkenyl, C₂₋₆alkynyl, C₁₋₆alkoxy, halogen, C₁₋₆haloalkyl, —N(R^{1a})₂, C₁₋₆acylamino, —NHSO₂C₁₋₆alkyl, —SO₂N(R^{1a})₂, —SO₂C₁₋₆alkyl, —COR^{1b}, nitro and cyano;
R^{1a} is independently hydrogen or C₁₋₆alkyl;
R^{1b} is —OR^{1a} or —N(R^{1a})₂;
R^{2a} and R^{2b} are (i) independently selected from the group consisting of hydrogen, C₁₋₁₀alkyl, —(CH2)ᵣN(R^{1a})₂, C₁₋₆hydroxyalkyl, —CH₂SH, —(CH₂)S(O)ₚMe, —(CH2)₃NHC(═NH)NH₂, (1H-indol-3-yl)methyl, (1H-indol-4-yl)methyl, —(CH2)ₘC(═O)R^{1b}, aryl and aryl C₁₋₃alkyl, said aryl groups optionally substituted with a group selected from the group consisting of hydroxyl, C₁₋₁₀alkyl, C₁₋₆alkoxy, halogen, nitro and cyano; (ii) R^{2a} is hydrogen and R^{2b} and R⁴ together are (CH₂)₃; (iii) R^{2a} and R^{2b} together are (CH₂)ₙ; or, (iv) R^{2a} and R^{2b} both are C₁₋₆ alkyl;
R³ is hydrogen, C₁₋₁₀ alkyl, C₁₋₁₀ haloalkyl, aryl or aryl-C₁₋₃ alkyl wherein said aryl is phenyl;
R⁴ is hydrogen, C₁₋₃ alkyl, or R^{2b} and R⁴ together are (CH₂)₃;
R⁶ is A, B, C or D wherein R⁸ is hydrogen or C₁₋₃ alkyl;
R⁵ and R⁷ is independently selected from hydrogen, C(═O)C₁₋₆alkyl, C(═O)R^{1b};
m is 0 to 3;
n is 4 or 5;
p is 0 to 2; and
r is 1 to 6;
or pharmaceutically acceptable salts thereof.

2. A method for treating dengue fever comprising administering to a patient in need thereof a compound of Formula Ia 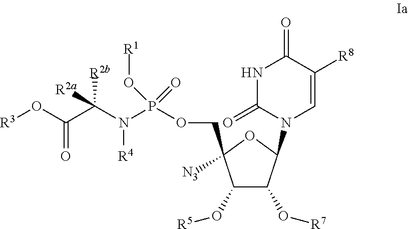
wherein:
R¹ is hydrogen, C₁₋₆haloalkyl, or aryl wherein said aryl is phenyl or naphthyl optionally substituted with one to three substituents independently selected from the group consisting of C₁₋₆alkyl, C₂₋₆alkenyl, C₂₋₆alkynyl, C₁₋₆alkoxy, halogen, C₁₋₆haloalkyl, —N(R^{1a})₂, C₁₋₆acylamino, —NHSO₂C₁₋₆alkyl, —SO₂N(R^{1a})₂, —SO₂C₁₋₆alkyl, —COR^{1b}, nitro and cyano;
R^{1a} is independently hydrogen or C₁₋₆alkyl;
R^{1b} is —OR^{1a} or —N(R^{1a})₂;
R^{2a} and R^{2b} are (i) independently selected from the group consisting of hydrogen, C₁₋₁₀alkyl, —(CH2)ᵣN(R^{1a})₂, C₁₋₆hydroxyalkyl, —CH₂SH, —(CH₂)S(O)ₚMe, —(CH2)₃NHC(═NH)NH₂, (1H-indol-3-yl)methyl, (1H-indol-4-yl)methyl, —(CH2)ₘC(═O)R^{1b}, aryl and aryl C₁₋₃alkyl, said aryl groups optionally substituted with a group selected from the group consisting of hydroxyl, C₁₋₁₀alkyl, C₁₋₆alkoxy, halogen, nitro and cyano; (ii) R^{2a} is hydrogen and R^{2b} and R⁴ together are (CH₂)₃; (iii) R^{2a} and R^{2b} together are (CH₂)ₙ; or, (iv) R^{2a} and R^{2b} both are C₁₋₆ alkyl;
R³ is hydrogen, C₁₋₁₀ alkyl, C₁₋₁₀ haloalkyl, aryl or aryl-C₁₋₃ alkyl wherein said aryl is phenyl;
R⁴ is hydrogen, C₁₋₃ alkyl, or R^{2b} and R⁴ together are (CH₂)₃;
R⁵ and R⁷ is independently selected from hydrogen, C(═O)C₁₋₆alkyl, C(═O)R^{1b};
R⁸ is hydrogen;
m is 0 to 3;
n is 4 or 5;
p is 0 to 2; and
r is 1 to 6;
or pharmaceutically acceptable salts thereof.

3. The method of claim 2 wherein:
R¹ is phenyl, naphthyl, or o-methoxyphenyl;
R^{2a} and R^{2b} are independently hydrogen, methyl, or benzyl;
R³ is methyl, ethyl, or benzyl;
R⁴ is H;
R⁵ and R⁷ are both H, —C(═O)Et, or —C(═O)Bu; and
R⁸ is H.

4. The method of claim 3 wherein:
R¹ is phenyl or naphthyl;
R^{2a} is hydrogen and R^{2b} is methyl;
R³ is ethyl or benzyl; and
R⁵ and R⁷ are both H or —C(═O)Et.

5. The method of claim 4 wherein:
R¹ is naphthyl;
R^{2a} is hydrogen and R^{2b} is methyl;
R³ is benzyl; and
R⁵ and R⁷ are both H.

6. The method of claim 3 wherein:
R¹ is naphthyl;
R^{2a} is H and R^{2b} is benzyl;
R³ is ethyl; and
R⁵ and R⁷ are both H.

7. The method of claim 3 wherein:
R¹ is naphthyl;
R^{2a} is H and R^{2b} is benzyl;
R³ is benzyl; and
R⁵ and R⁷ are both H.

8. The method of claim 1 wherein:
R¹ is phenyl;
R^{2a} is H and R^{2b} is methyl;
R³ is benzyl;
R⁵ is H;
R⁶ is C; and
R⁷ is H.

9. A method for treating dengue fever comprising administering to a patient in need thereof a compound selected from the group consisting of:
(S)-2-[[(2R,3S,4R,5R)-5-(6-Amino-purin-9-yl)-2-azido-3,4-dihydroxy-tetrahydro-furan-2-ylmethoxy]-(naphthalen-1-yloxy)-phosphorylamino]-propionic acid ethyl ester;
(S)-2-{[(2R,3S,4R,5R)-5-(6-Amino-purin-9-yl)-2-azido-3,4-dihydroxy-tetrahydro-furan-2-ylmethoxy]-phenoxy-phosphorylamino}-propionic acid benzyl ester;
(S)-2-{[(2R,3S,4R,5R)-5-(4-Amino-2-oxo-2H-pyrimidin-1-yl)-2-azido-3,4-dihydroxy-tetrahydro-furan-2-ylmethoxy]-phenoxy-phosphorylamino}-propionic acid methyl ester;
Pentanoic acid (2R,3S,4R,5R)-5-(4-amino-2-oxo-2H-pyrimidin-1-yl)-2-azido-2-[((S)-1-benzyloxycarbonyl-ethylamino)-(2-methoxy-phenoxy)-phosphoryloxymethyl]-4-pentanoyloxy-tetrahydro-furan-3-yl ester;
(S)-2-[[(2R,3S,4R,5R)-5-(4-Amino-2-oxo-2H-pyrimidin-1-yl)-2-azido-3,4-dihydroxy-tetrahydro-furan-2-ylmethoxy]-(naphthalen-1-yloxy)-phosphorylamino]-propionic acid benzyl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-propionic acid benzyl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-pentanedioic acid diethyl ester;
(S)-2-{[(2R,3S,4R,5R)-2-Azido-5-(2,4-dioxo-3,4-dihydro-2H-pyrimidin-1-yl)-3,4-bis-propionyloxy-tetrahydro-furan-2-ylmethoxy]-phenoxy-phosphorylamino}-propionic acid ethyl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-3-phenyl-propionic acid benzyl ester; and
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-3-phenyl-propionic acid ethyl ester.

10. The method of claim 1 further comprising administering at least one other antiviral agent.

11. A compound selected from the group consisting of:
Pentanoic acid (2R,3S,4R,5R)-5-(4-amino-2-oxo-2H-pyrimidin-1-yl)-2-azido-2-[((S)-1-benzyloxycarbonyl-ethylamino)-(2-methoxy-phenoxy)-phosphoryloxymethyl]-4-pentanoyloxy-tetrahydro-furan-3-yl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-pentanedioic acid diethyl ester;
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-3-phenyl-propionic acid benzyl ester; and
(S)-2-[[(2R,3S,4R,5R)-2-Azido-5-(2,4-dioxo-3,4-dihydro-2H-pyrimidin-1-yl)-3,4-dihydroxy-tetrahydro-furan-2-ylmethoxy]-(naphthalen-1-yloxy)-phosphorylamino]-3-phenyl-propionic acid ethyl ester.
